SEQUENCE LISTING (19 sequences)

SEQ ID NO: 1 - DNA, 21 bp, Artificial Sequence
  Sulfate Reducing Bacteria
ccngtncgyaccggtaaatgg

SEQ ID NO: 2 - DNA, 18 bp, Artificial Sequence
  Sulfate Reducing Bacteria
ccngtnggcgcntggttc

SEQ ID NO: 3 - DNA, 17 bp, Artificial Sequence
  Sulfate Reducing Bacteria
gagaacttgtgnccgga

SEQ ID NO: 4 - DNA, 15 bp, Artificial Sequence
  Sulfate Reducing Bacteria
cayacccagggytgg

SEQ ID NO: 5 - DNA, 14 bp, Artificial Sequence
  Sulfate Reducing Bacteria
cacacbaaggdtgg

SEQ ID NO: 6 - DNA, 15 bp, Artificial Sequence
  Sulfate Reducing Bacteria
cayacbcaaggctgg

SEQ ID NO: 7 - DNA, 15 bp, Artificial Sequence
  Sulfate Reducing Bacteria
catacdcaggghtgg

SEQ ID NO: 8 - DNA, 15 bp, Artificial Sequence
  Sulfate Reducing Bacteria
cacacdcagggrtgg

SEQ ID NO: 9 - DNA, 15 bp, Artificial Sequence
  Sulfate Reducing Bacteria
cacacdcagggytgg

SEQ ID NO: 10 - DNA, 15 bp, Artificial Sequence
  Sulfate Reducing Bacteria
catacccagggntay

SEQ ID NO: 11 - DNA, 15 bp, Artificial Sequence
  Sulfate Reducing Bacteria
catacwcaggghtat

SEQ ID NO: 12 - DNA, 21 bp, Artificial Sequence
  Sulfate Reducing Bacteria
ccatacnggrtaccakgcrcg

SEQ ID NO: 13 - DNA, 19 bp, Artificial Sequence
  Sulfate Reducing Bacteria
ggaagtcttcccangcttc

SEQ ID NO: 14 - DNA, 21 bp, Artificial Sequence
  Sulfate Reducing Bacteria
tgggaagabttcctvgacatg

SEQ ID NO: 15 - DNA, 17 bp, Artificial Sequence
  Sulfate Reducing Bacteria
gtgtarcagttrccrca

SEQ ID NO: 16 - DNA, 26 bp, Artificial Sequence
  Sulfate Reducing Bacteria
cagatcatgatcaayggtgartcyta

SEQ ID NO: 17 - DNA, 19 bp, Artificial Sequence
  Sulfate Reducing Bacteria
cgaagactactgngnvacc

SEQ ID NO: 18 - DNA, 21 bp, Artificial Sequence
  Sulfate Reducing Bacteria
cgcccacdccgtcngcvcagg

SEQ ID NO: 19 - DNA, 15 bp, Artificial Sequence
  Sulfate Reducing Bacteria
tsaacatgtgcggcg

(19) United States
(12) Patent Application Publication
(10) Pub. No.: US 2016/0265035 A1
(43) Pub. Date: Sep. 15, 2016

(54) PCR AMPLIFICATION METHODS, PRIMERS, AND PROBES FOR DETECTING AND QUANTIFYING SULFATE-REDUCING BACTERIA

(71) Applicant: InstantLabs Medical Diagnostics Corporation, Baltimore, MD (US)

(72) Inventors: Cyrstal Lee, Houston, TX (US); Neil Sharma, Rockville, MD (US); Angela Reeves, Chicago, IL (US)

(73) Assignee: InstantLabs Medical Diagnostics Corporation, Baltimore, MD (US)

(21) Appl. No.: 15/066,321

(22) Filed: Mar. 10, 2016

Related U.S. Application Data
(60) Provisional application No. 62/132,122, filed on Mar. 12, 2015.

(51) Int. Cl. C12Q 1/68

(52) U.S. Cl. CPC C12Q 1/689; C12Q 2600/158

(57) ABSTRACT

At least one nucleic acid from a sulphate-reducing bacterium may be extracted from a sample and may be amplified by a PCR amplification method in the presence of at least one primer to form an amplification product. The primer(s) may include a sequence essentially identical to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof. A probe may hybridize with the amplification product from the PCR amplification method where the probe includes a sequence essentially identical to SEQ ID NO: 16, SEQ ID NO: 17, SEQ ID NO: 18, SEQ ID NO: 19, and mixtures thereof.

### TECHNICAL FIELD

The present invention relates to amplifying, optionally detecting and optionally quantifying sulfate-reducing bacteria, and more specifically relates to rapid amplification of sulfate-reducing bacteria using real-time quantitative polymerase chain reactions (qPCR).

### BACKGROUND

The presence of sulfate-reducing bacteria in many environments is undesirable, particularly in concentrations sufficient to cause significant corrosion of metals with aqueous solutions, including fresh and seawaters, having the sulfate-reducing bacteria (SRB) therein. SRBs are present in a variety of environments, including oil- and gas-bearing formations, soils, and wastewater. SRBs are also present in the gut of ruminant animals, particularly domestic animals (cattle) used as protein sources for human consumption.

Sulfate-reducing bacteria, such as members of the genera Desulfovibrio and Desulfotomaculum, may reduce sulfate and/or sulfite under suitable conditions (e.g. anaerobic conditions) and generate hydrogen sulfide, an odiferous, and poisonous gas. In addition, the sulfate-reducing bacteria may contact metals thereby causing corrosion to the metal, such as metal structures and conduits. “Sulfate-reducing bacteria” is defined herein to be bacteria capable of reducing sulfate to sulfite and/or bacteria capable of reducing sulfite to sulfide, regardless of the taxonomic group of the bacteria.

Traditionally, the monitoring of microbial populations has employed microbial growth tests where a sample is diluted to various levels and used to inoculate microbial growth media designed to favor the growth of various types of bacteria. After days to several weeks of incubation, the growth tests are scored based on the presence or absence of growth in these various microbiological media. Unfortunately, as numerous researchers show, only about 0.1% to about 10% bacteria from environmental samples can actually grow in an artificial medium, and a significant portion of bacteria growing in the media are not actually the target bacteria. Therefore, growth tests are unable to provide the accurate quantification of target bacteria in the samples. In addition, obtaining results from a serial dilution assay may take as long as three to four weeks.

To circumvent problems associated with such growth-based methods, many culture-independent genetic techniques have been developed in the past decade to detect pathogens in the field of medicine, the food industries, the oil and gas industries, and the like. Because many ecosystems have a relatively low abundance of microorganisms, the polymerase chain reaction (PCR) has been widely used to amplify the genetic signals of microbes in complex environmental samples. However, traditional PCR-based methods are significantly biased by amplification efficiency and the depletion of PCR reagents.

Real-time quantitative PCR (qPCR) may be used to detect and quantify a number of microorganisms. Quantitative PCR has also been used to determine the abundance of microorganisms in many different types of complex environmental samples, such as sediments, water, wastewater, and marine samples. qPCR may provide more accurate and reproducible quantification of microorganisms because qPCR quantifies the PCR products during the logarithmic phase of the reactions, which does not occur during traditional PCR methods. Moreover, qPCR offers a dynamic detection range of six orders of magnitude or more, does not need post-PCR manipulation, and has the capability of high throughput analysis.

Digital PCR (dPCR) may be used to directly quantify and clonally amplify nucleic acids including DNA, cDNA, and/or RNA. dPCR may be more precise method than PCR and/or qPCR. Traditional PCR carries out one reaction per single sample. dPCR may carry out a single reaction within a sample, but the sample may be separated into a large number of partitions, and the reaction may be individually carried out within each partition. The separation may allow for a more reliable collection and a more sensitive measurement of nucleic acid amounts within the sample. dPCR may be useful for studying variations in gene sequences, such as copy number variants, point mutations, and the like, and dPCR may be routinely used for clonal amplification of samples for “next-generation sequencing.”

It would be desirable to have a method of detecting and optionally quantifying sulfate-reducing bacteria within a sample that is cost-effective and may occur in real time.

### SUMMARY

There is provided, in one form, a PCR amplification method. The method may include amplifying at least one nucleic acid of at least one sulfur-reducing bacteria in the presence of at least one primer to form an amplification product. The nucleic acid(s) is extracted from a sample prior to amplifying the nucleic acid(s). The primer(s) may include an essentially identical sequence, such as but not limited to, SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof.

An alternative non-limiting embodiment of the method may also include hybridizing the amplification product with a probe specific for a fragment of an alpha subunit of an APS gene and optionally detecting a presence of hybridization and a degree of hybridization. The presence of hybridization may indicate the presence of the sulfate-reducing bacteria. The degree of hybridization may enumerate the sulfate-reducing bacteria. In a non-limiting embodiment, the hybridization may be considered a fluorescent in situ hybridization (i.e. FISH).

In another non-limiting embodiment, a primer for PCR amplification of at least one nucleic acid of at least one sulfur-reducing bacteria is provided. The primer may include, but is not limited to, a nucleotide sequence essentially identical to SEQ ID:1, SEQ ID:2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof.

In another non-limiting embodiment, a probe for hybridizing with a PCR amplification product is provided. The probe may include, but is not limited to, a nucleotide sequence essentially identical to SEQ ID NO:16, SEQ ID NO:17, SEQ ID NO:18, SEQ ID NO:19, and mixtures thereof.

The primers, probes, and PCR amplification methods may be useful for detecting sulfur-reducing bacteria within a sample.

### BRIEF DESCRIPTION OF THE DRAWINGS

In order to more fully understand the drawings referred to in the detailed description, a brief description of each drawing is presented here:

FIGS. 1-11 (SEQ ID NO:1 through SEQ ID NO:11) represent the nucleotide sequences of a forward primer usable to detect sulfur-reducing bacteria;

FIGS. 12-15 (SEQ ID NO:12 through SEQ ID NO: 15) represent the nucleotide sequence of a reverse primer usable to detect sulfur-reducing bacteria;

FIGS. 16-19 (SEQ ID NO:16 through SEQ ID NO: 19) represent the nucleotide sequence of a probe usable to detect sulfur-reducing bacteria; and

FIG. 20 represents a non-limiting example of a restriction map of a plasmid pCI BSR used as an internal control, obtained from a plasmid pUC19.

### DETAILED DESCRIPTION

It has been discovered that a polymerase chain reaction (PCR) amplification method may be used to amplify at least one nucleic acid of at least one sulfur-reducing bacteria (SRB) in the presence of at least one primer to form an amplification product. This method of amplification, optional detection and optional quantification of SRBs present in a particular sample is much quicker than previous methods of detecting SRBs. For example, the PCR amplification methods described below may occur in an amount of time less than about a 7 calendar days, alternatively less than 2 calendar days, or less than 24 hours in another non-limiting embodiment. In yet another non-limiting embodiment, the PCR amplification methods may occur in less than 8 hours.

In an alternative embodiment, the method of amplification, optional detection and optional quantification may occur in an amount of time less than about a 7 calendar days, alternatively less than 2 calendar days, or less than 24 hours in another non-limiting embodiment. In yet another non-limiting embodiment, the PCR amplification, optional detection and optional quantification methods may occur in less than 8 hours.

‘Amplification’ as defined herein refers to any in vitro method for increasing the number of copies of a nucleotide sequence with the use of a DNA polymerase, such as a PCR method of amplification in a non-limiting embodiment. PCR amplification methods may include from about 10 cycles independently to about 50 cycles of denaturization and synthesis of a DNA molecule.

Prior to amplifying the nucleic acid(s) of the SRBs, the nucleic acids must first be extracted from a sample. The sample may be in any form necessary to obtain the sulfur-reducing bacteria, such as a fluid sample containing the SRB, a ground-up version of a tissue where it would be beneficial to determine whether the SRB are present in the tissue, and the like. In an alternative embodiment, a surface and/or surface solids suspected of having SRB contamination may be swabbed, and the swab may be placed in a fluid to obtain the SRB fluid sample. Non-limiting examples of a sample may be a food product, an animal tissue, a human tissue, a water sample, a lab surface, a metal surface, a paper mill industry surface, a waste water within a wastewater treatment facility, a sample from the paint industry, and combinations thereof.

The nucleic acid may be or include, DNA, RNA (e.g. mRNA), and combinations thereof. The nucleic acid(s) from the sulphate-reducing bacteria within the sample may be extracted from the sample prior to amplifying the nucleic acid(s). Such extraction techniques of the nucleic acids from the sample may be carried out by standard techniques, which are well known to persons skilled in the art.

A non-limiting example of an extraction technique may be or include using the QIAamp Tissue Kit (QIAGEN, Hilden, Germany), the MP Bio Soil DNA kit, and the like. DNA from the SRBs may be extracted from a sample using the QIAamp Tissue Kit by performing the following method:
Centrifuge 1 mL of sample for 30 minutes at 15,000 rpm, and then remove the supernatant.
Add 200 μL of INSTAGENE™ template (Bio-rad laboratories, Hercules, Calif.) (previously homogenized) to the pellet.
Vortex the mixture for about 30 minutes at 56° C.
Vortex the mixture for 8 minutes at 100° C.
Centrifuge the sample for about 2 minutes at 12,000 rpm.
Remove about 20 μL of the supernatant to directly use in a PCR reaction.

Another non-limiting example of an extraction technique may be or include using the MP Bio Soil DNA kit. The DNA from the SRBs may be extracted from a sample by performing the following method:
Add up to 500 mg of a soil sample to a Lysing Matrix E tube.
Add 978 μl sodium phosphate buffer to the sample in the lysing matrix E tube.
Add 122 μl MT Buffer (an alkaline solution with surfactant to lyse a cell) to the lysing matrix E tube.
Homogenize the mixture in a FASTPREP™ Instrument for 40 seconds at a speed setting of about 6.0.
Centrifuge the mixture at 14,000×g for 5-10 minutes to pellet the debris; the centrifugation may be extended to about 15 minutes to enhance elimination of excessive debris from large samples, or from cells with complex cell walls.
Transfer the supernatant to a clean 2.0 mL microcentrifuge tube.
Add 250 μl of a protein precipitation solution (PPS) to the microcentrifuge tube and shake the tube by hand about 10 times.
Centrifuge the microcentrifuge tube at 14,000×g for about 5 minutes to pellet the precipitate.
Transfer the supernatant to a clean 15 mL tube. A 2.0 mL microcentrifuge tube may be used at this step, but better mixing and DNA binding may occur in a larger tube.
Resuspend the binding matrix suspension (a solution of small silicon beads) and add 1.0 mL of the resuspended binding matrix suspension to the supernatant within the 15 mL tube.
Place the 15 mL tube on a rotator or invert the 15 mL tube by hand for about 2 minutes to allow binding of the DNA with the binding matrix.
Place the 15 mL tube on a rack for about 3 minutes to allow settling of the binding matrix.
Remove and discard 500 μL of the supernatant being careful to avoid the settled binding matrix.
Resuspend the settled binding matrix in the remaining amount of the supernatant and transfer approximately 600 μL of the mixture to a SPIN™ Filter and centrifuge at 14,000×g for 1 minute.
Empty the catch tube (the catch tube ‘catches’ the portion of the mixture that goes through the filter) and add the remaining mixture from the resuspension of the settled binding matrix within the supernatant, from the above step, to the SPIN™ Filter and centrifuge at 14,000×g for 1 minute.
Empty the catch tube again.
Add 500 μL prepared SEWS-M (a wash buffer that contains ethanol) and gently resuspend the remaining pellet using the force of the liquid from the pipet tip (ensure that ethanol has been added to the Concentrated SEWS-M).
Centrifuge the resuspended pellet in SEWS-M at 14,000×g for 1 minute.
Empty the catch tube and replace.
Without any addition of liquid, centrifuge the resuspended pellet in SEWS-M a second time at 14,000×g for 2 minutes to “dry” the matrix of residual wash solution.
Discard the catch tube and replace with a new, clean catch tube.
Air dry the SPIN™ Filter for about 5 minutes at room temperature (about 65° F. to about 80° F.).
Gently resuspend the Binding Matrix pellet (the portion above or on top of the SPIN filter) in 50-100 μl of DES (DNase/Pyrogen-Free Water). To avoid over-dilution of the purified DNA, use the smallest amount of DES required to resuspend the Binding Matrix pellet. Yields may be increased by incubation for 5 minutes at 55° C. in a heat block or water bath.
Centrifuge the resuspended Binding Matrix pellet in DES at 14,000×g for 1 minute to bring eluted DNA into a clean catch tube; discard the SPIN filter.
The remaining DNA may be amplified by PCR amplification techniques and other downstream applications; store at about a temperature ranging from about −20° C. to about 4° C. until the extracted nucleic acids are ready to be amplified.

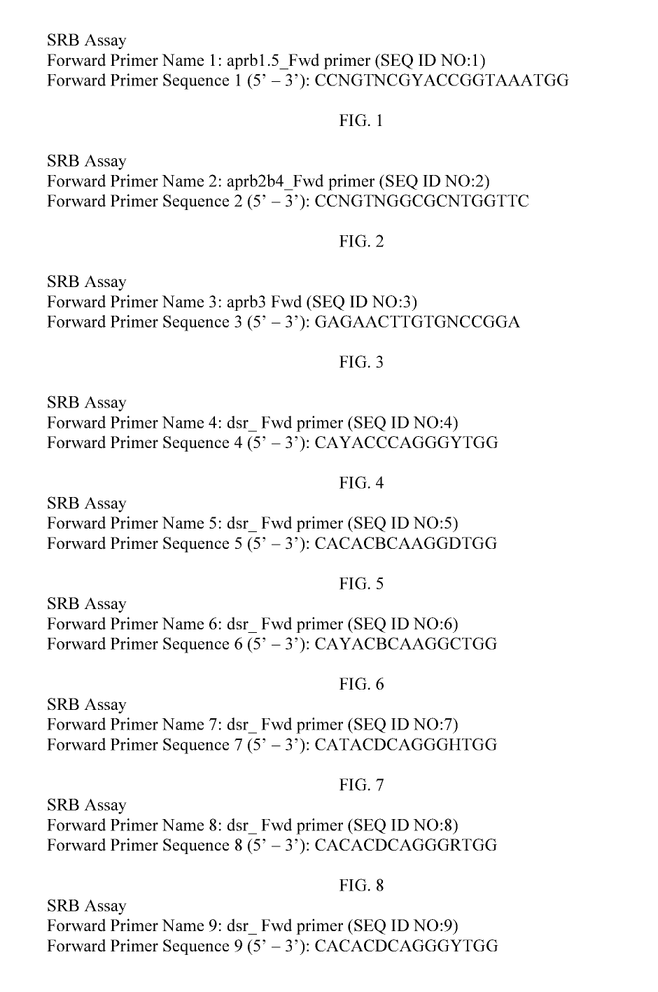

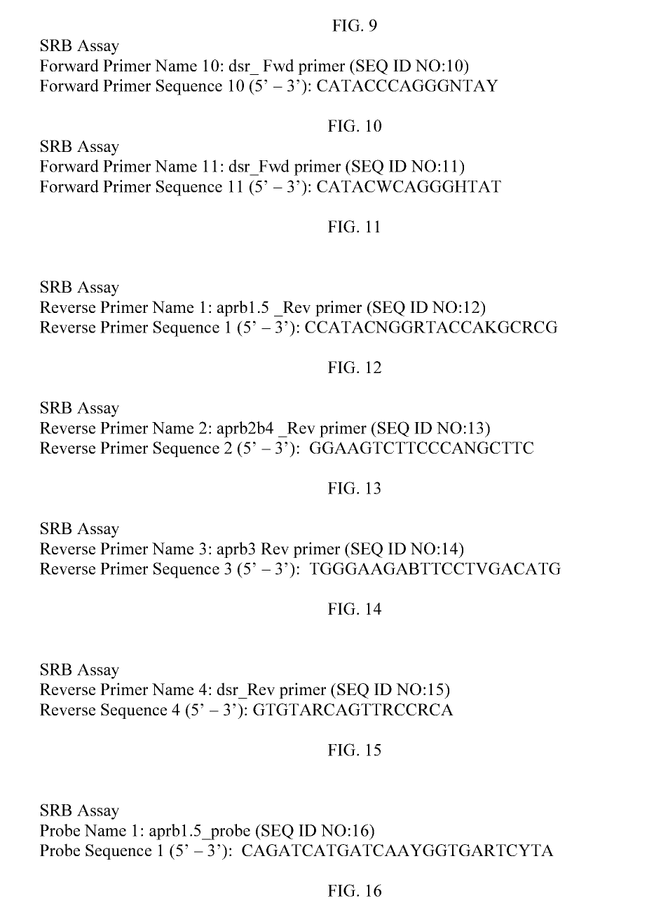

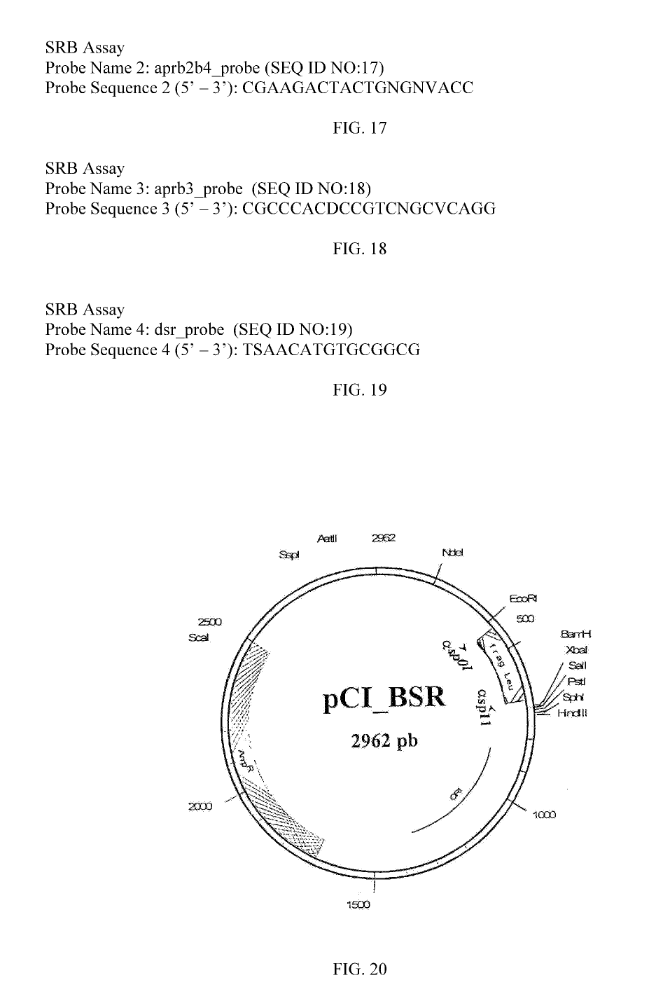

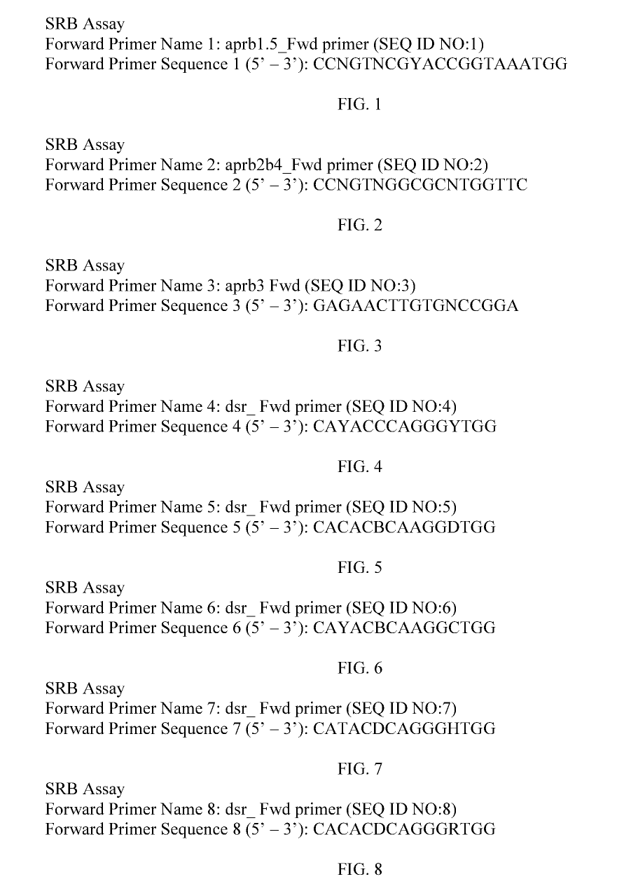

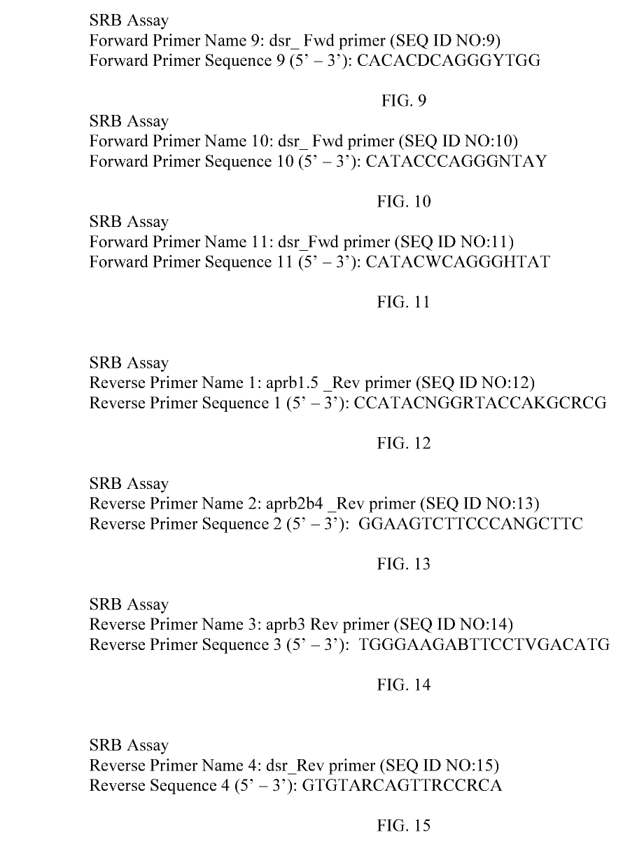

Once the nucleic acid(s) are extracted, the nucleic acid(s) may be combined with at least one primer in a reaction well to start and/or improve the amplification of the nucleic acids using a PCR method. The primer(s) may be or include a sequence that is essentially identical to SEQ ID NO:1 through SEQ ID: 15 (FIGS. 1-15), and mixtures thereof. Any of the sequences identified as SEQ ID NO:1 through SEQ ID NO: 11, or a combination thereof, may act as the forward primer. Any of the sequences identified as SEQ ID NO:12 through SEQ ID: 15, or a combination thereof, may act as the reverse primer. The primer(s) may be specific for amplification of at least a fragment of an alpha subunit of an APS reductase gene. Alternatively, the primer(s) may include an oligonucleotide from the alpha subunit of the APS reductase gene.

APS reductase (also known as Adenylylsulfate Reductase) allows the reduction of adenosine phosphosulfate (APS—a product of the activation of sulfate by ATP sulfurylase). APS reductase is a cytoplasmic enzyme containing two subunits (alpha and beta) known to be involved only in the anaerobic respiration of sulfate. This enzyme may not be present in non-sulfate-reducing organisms, since it is not involved in the assimilatory reduction that allows the incorporation of sulfur into various molecules necessary for life, such as amino acids and vitamins. Therefore, detecting fragments of the gene(s) that may code for APS reductase may allow for the detection of a sulfur-reducing bacteria.

“Essentially identical” is defined herein to mean that the sequence of the oligonucleotide is identical to at least one of the sequences (i.e. SEQ ID NO: 1 through SEQ ID NO:15), or that the oligonucleotide sequence differs from one of the sequences without affecting the capacity of these sequences to hybridize with the gene for the alpha subunit of APS reductase. A sequence that is “essentially identical” to SEQ ID NO:1 through SEQ ID NO:15 may differ therefrom by a substitution of one or more bases or by deletion of one or more bases located at the ends of the sequence, or alternatively by addition of one or more bases at the ends of the sequence.

‘Primer’ as defined herein refers to a single-stranded oligonucleotide that is extended by covalent bonding of nucleotide monomers during amplification or polymerization of a nucleic acid molecule. ‘Oligonucleotide’ as defined herein refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

The components for a PCR method of amplification must be added to a reaction well prior to performing the PCR method of amplification. In a non-limiting embodiment, the components may include the forward primer (also known as a sense primer), the reverse primer (also known as an antisense primer), PCR buffer, dNTP, DNA, Taq DNA polymerase, water, and combinations thereof. The amounts of the components within a reaction well are very well known to those skilled in the art, and the components within the reaction well may vary depending on the amounts of the other components present.

dNTPs are deoxynucleotide triphosphates included in a solution for purposes of PCR amplification. Stock dNTP solutions may have a pH of about 7, and the stability of dNTPs during repeated cycles of PCR may leave about 50% of the dNTPs remaining after about 50 PCR cycles. The concentration of each of the four dNTPs in solution ranges from about 20 μM to about 200 μM. Taq DNA polymerase is an enzyme used to replicate the DNA during the amplification where the enzyme may withstand the protein-denaturing conditions required for PCR methods of amplification.

PCR methods of amplification require particular conditions of temperature, reaction time, and optionally the presence of additional agents and/or reagents that are necessary for the fragment of the gene for the alpha subunit of APS reductase, to which the primers as defined above have hybridized, to be copied identically. Such conditions are well known to those skilled in the art. An average PCR program runs about 30 to about 65 cycles, but more or less cycles may be used depending on the conditions of the DNA, desired number of amplification products, time constraints, etc.

A non-limiting example of a PCR program having 42 total cycles may run where the first cycle runs for about 3 minutes at about 95 C, and cycles 2-6 run for about 1 minute at about 94° C. then 30 seconds at 54° C. then 10 seconds at 72° C. Cycles 7-41 may run for 30 seconds at 94° C., and then 10 seconds at 72° C. Cycle 42 may run for 5 minutes at 72 C and then held at 4 C until the reaction wells are removed from the PCR device. Computer processing may be used to analyze the crude amplification products. The PCR program mentioned above is strictly a non-limiting example and should not be deemed to limit the invention here.

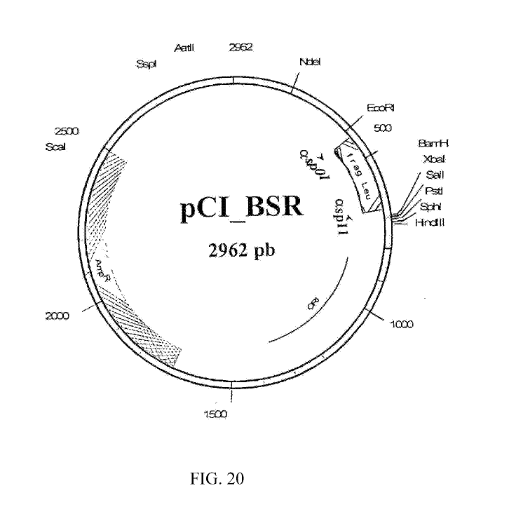

An internal amplification control may be used in order to avoid an ambiguous interpretation of negative results of the PCR amplification method. For example, an absence of amplification by PCR may be due to problems of inhibition of the reaction, or to the absence of a target, i.e. the absence of DNA from the sulfur-reducing bacteria. The internal control may be a plasmid (FIG. 20) including oligonucleotide sequences that allows the amplification of a fragment of the APS reductase gene (289 base pairs) when no target is present in the sample. Thus, the presence of a fragment of 289 base pairs, without a fragment size having a different number of base pairs of the selected target, may indicate the functioning of the reaction and the absence of a specific target, i.e. the sulfate-reducing bacteria, from the sample. Also, the sequence intercalated between the primers asp01 and asp11 in the internal control differs by its size but also by its sequence (Leu2 gene), which makes it possible not to confuse the amplification of the internal control with the specific amplification of a fragment of the APS reductase gene whether the PCR analysis is performed on agarose gel or by hybridization. Such oligonucleotide sequences specific for a fragment of the APS reductase gene may be chosen in particular from SEQ ID NO:1 through SEQ ID NO: 15, and mixtures thereof.

When added in a limited concentration to the PCR reaction mixture, the plasmid allows the amplification of a DNA fragment when no specific target is present in the sample. This indicates the functioning of the reaction and the absence of a specific target, i.e. sulfate-reducing bacteria.

The amplification of at least one fragment of the APS reductase gene may allow for the detection of the fragment of the APS reductase gene, such as the gene for the alpha subunit of the APS reductase in a non-limiting embodiment. The gene amplification products may be optionally subjected to hybridization with a probe that is specific for a fragment of the gene for the alpha subunit of the APS reductase where the probe may be labeled in a detectable manner, such as but not limited to fluorescent labeling, radioactive labeling, chemiluminescent labeling, enzymatic labeling, and combinations thereof. ‘Gene’ is defined herein to mean a DNA sequence containing information required for expression of a polypeptide or protein.

Hybridizing the amplification product with a probe also requires particular conditions of temperature, reaction time, and preventing the hybridization of the oligonucleotide with sequences other than the gene for the alpha subunit of APS reductase. In a non-limiting example, the hybridization temperature may range from about 55° C. to about 65° C. The reaction time for the hybridization may range from about 0 seconds independently to about 60 seconds. The hybridization buffer may be a solution with a high ionic strength, such as a 6×SSC solution in a non-limiting example. As used herein with respect to a range, “independently” means that any threshold may be used together with another threshold to give a suitable alternative range.

The probe is a fragment of DNA used to detect the presence of nucleotide sequences that are complementary to the sequence in the probe. The probe hybridizes to a single-stranded nucleic acid, whose base sequence allows probe-target base pairing due to complementarity between the probe and the target (e.g. single-stranded DNA from the sulfur-reducing bacteria). First, the probe may be denatured (by heating or under alkaline conditions, such as exposure to sodium hydroxide) into single stranded DNA (ssDNA) and then hybridized to the target ssDNA, i.e. by Southern blotting in a non-limiting example. The hybridization may occur when the target ssDNA and probe are immobilized on a membrane (e.g. a gel) or in situ. ‘Target’ as used herein refers to DNA of the sulfur-reducing bacteria.

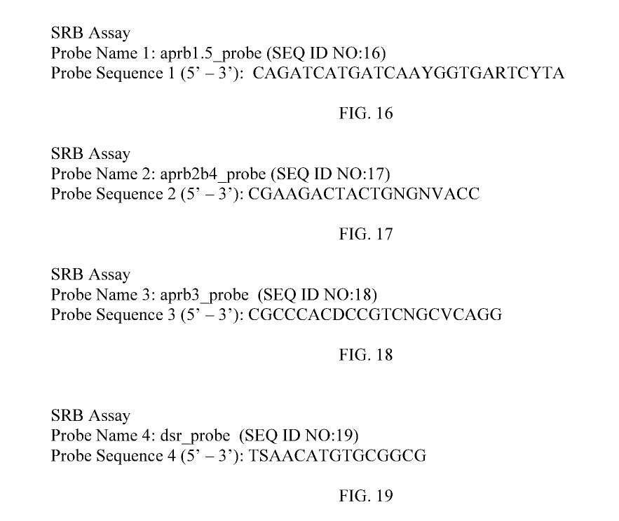

The resulting amplification product may be hybridized with a probe specific for a fragment of an alpha subunit of an APS gene. The probe may have a nucleotide sequence that specifically hybridizes to the complement of a nucleotide sequence essentially identical to at least one of SEQ ID NO: 16 through SEQ ID NO:19 (FIGS. 16-19). In a non-limiting embodiment, the probe may include a dye, such as those sold as QUASAR™ (e.g. QUASAR™ 670), BHQ PLUS, or combinations thereof.

A presence of hybridization and a degree of hybridization may be detected. The presence of hybridization may indicate the presence of the sulfate-reducing bacteria, and the degree of hybridization may enumerate the sulfate-reducing bacteria.

In a non-limiting embodiment, the method may be performed by
amplifying at least one nucleic acid of at least one sulfur-reducing bacteria in the presence of at least one primer to form an amplification product where the nucleic acid(s) are extracted from a sample prior to amplifying the nucleic acid(s). The primer(s) may include an oligonucleotide having a nucleotide sequence essentially identical to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof;
optionally hybridizing the amplification product with a probe having a nucleotide sequence that is essentially identical to SEQ ID NO: 16, SEQ ID NO: 17, SEQ ID NO: 18, SEQ ID NO: 19, and mixtures thereof; and
optionally detecting the hybridization complex formed between the product of amplification and the probe to indicate the presence of sulphate-reducing bacteria in the sample.

The type of sulfur-species bacteria that may be detected by the methods may be or include, but are not limited to, Desuffovibrio vulgaris, Desuffovibrio desuffuricans, Desuffovibrio aespoeensis, Thermodesuffobium narugense, Desuffotomaculum carboxydivorans, Desuffotomaculum ruminis, Desuffovibrio africanus, Desuffovibrio hydrothermalis, Desuffovibrio piezophilus, Desuffobacterium corrodens, Sulfate-reducing bacterium QLNR1, Desuffobacterium catecholicum, Desuffobacterium catecholicum, Desuffobulbus marinus, Desuffobulbus, Desuffobulbus propionicus, Desuffocapsa thiozymogenes, Desuffocapsa suffexigens, Desufforhopalus vacuolatus, Desufforhopalus, Desuffofustis glycolicus strain, Desufforhopalus singaporensis, Desuffobacterium, Desuffobacterium zeppelinii strain, Desuffobacterium autotrophicum, Desuffobacula phenolica, Desuffobacula toluolica Tol2, Sulfate-reducing bacterium JHA1, Desuffospira joergensenii, Desuffobacter, Desuffobacter postgatei, Desuffotignum, Desuffotignum balticum, Desufforegula conservatrix, Desuffocella, Desuffobotulus sapovorans, Desuffofrigus, Desuffonema magnum, Desuffonema limicola, Desuffobacterium indolicum, Desuffosarcina variabilis, Desuffatibacillum, Desuffococcus multivorans, Desuffococcus, Desuffonema ishimotonii, Desuffococcus oleovorans Hxd3, Desuffococcus niacini, Desuffotomaculum, Desuffotomaculum nigrificans, Desuffotomaculum ruminis, Desuffotomaculum halophilum, Desuffotomaculum acetoxidans, Desuffotomaculum gibsoniae, Desuffotomaculum sapomandens strain, Desuffotomaculum thermosapovorans, Desuffotomaculum, Desuffotomaculum geothermicum, Desuffotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

In the foregoing specification, the invention has been described with reference to specific embodiments thereof, and has been described as effective in providing methods and compositions for PCR amplification methods, and primers and/or probes useful therefor. However, it will be evident that various modifications and changes can be made thereto without departing from the broader spirit or scope of the invention as set forth in the appended claims. Accordingly, the specification is to be regarded in an illustrative rather than a restrictive sense. For example, specific samples, nucleic acids, forward primers, reverse primers, probes, PCR cycles, sulfur-reducing bacteria, internal controls (plasmids), and the like falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the PCR amplification method may consist of or consist essentially of amplifying at least one nucleic acid of at least one sulfur-reducing bacteria in the presence of at least one primer to form an amplification product; the nucleic acid(s) is extracted from a sample prior to amplifying the nucleic acid(s); the primer(s) may include an essentially identical nucleotide sequence to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof.

The primer for PCR amplification of at least one nucleic acid of at least one sulfur-reducing bacteria may consist or consist essentially of a nucleotide sequence essentially identical to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof.

The probe for hybridizing with a PCR amplification product may consist or consist essentially of a nucleotide sequence essentially identical to SEQ ID NO: 16, SEQ ID NO: 17, SEQ ID NO: 18, SEQ ID NO: 19, and mixtures thereof.

The words “comprising” and “comprises” as used throughout the claims, are to be interpreted to mean “including but not limited to” and “includes but not limited to”, respectively.

What is claimed is:

1. A PCR amplification method comprising:
amplifying at least one nucleic acid of at least one sulfur-reducing bacteria in the presence of at least one primer to form an amplification product; wherein the at least one nucleic acid is extracted from a sample prior to amplifying the at least one nucleic acid; wherein the at least one primer comprises an essentially identical sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 12, SEQ ID NO: 13, SEQ ID NO: 14, SEQ ID NO: 15, and mixtures thereof.

2. The method of claim 1 further comprising hybridizing the amplification product with a probe specific for a fragment of an alpha subunit of an APS gene.

3. The method of claim 2, wherein the probe has a nucleotide sequence essentially identical to SEQ ID NO:16, SEQ ID NO:17, SEQ ID NO:18, SEQ ID NO:19, and mixtures thereof.

4. The method of claim 2 further comprising detecting a presence of hybridization and a degree of hybridization; wherein the presence of hybridization indicates the presence of the at least one sulfate-reducing bacteria; and wherein the degree of hybridization enumerates the at least one sulfate-reducing bacteria.

5. The method of claim 2, wherein the probe is detectably labeled.

6. The method of claim 1, wherein the at least one primer is specific for amplification of at least a fragment of an alpha subunit of an APS reductase gene.

7. The method of claim 1, wherein the sample is selected from the group consisting of a food product, an animal tissue, a human tissue, a water sample, a lab surface, a metal surface, a paper mill industry sample, a waste water sample from a wastewater treatment facility, a sample from the paint industry, and combinations thereof.

8. The method of claim 1, further comprising extracting the at least one nucleic acid from the sample prior to amplifying at least one nucleic acid.

9. The method of claim 1, wherein the at least one sulfur-species bacteria is selected from the group consisting of Desuffovibrio vulgaris, Desuffovibrio desuffuricans, Desuffovibrio aespoeensis, Thermodesuffobium narugense, Desuffotomaculum carboxydivorans, Desuffotomaculum ruminis, Desuffovibrio africanus, Desuffovibrio hydrothermalis, Desuffovibrio piezophilus, Desuffobacterium corrodens, Sulfate-reducing bacterium QLNR1, Desuffobacterium catecholicum, Desuffobacterium catecholicum, Desuffobulbus marinus, Desuffobulbus, Desuffobulbus propionicus, Desuffocapsa thiozymogenes, Desuffocapsa suffexigens, Desufforhopalus vacuolatus, Desufforhopalus, Desuffofustis glycolicus strain, Desufforhopalus singaporensis, Desuffobacterium, Desuffobacterium zeppelinii strain, Desuffobacterium autotrophicum, Desuffobacula phenolica, Desuffobacula toluolica Tol2, Sulfate-reducing bacterium JHA1, Desuffospira joergensenii, Desuffobacter, Desuffobacter postgatei, Desuffotignum, Desuffotignum balticum, Desufforegula conservatrix, Desuffocella, Desuffobotulus sapovorans, Desuffofrigus, Desuffonema magnum, Desuffonema limicola, Desuffobacterium indolicum, Desuffosarcina variabilis, Desuffatibacillum, Desuffococcus multivorans, Desuffococcus, Desuffonema ishimotonii, Desuffococcus oleovorans Hxd3, Desuffococcus niacini, Desuffotomaculum, Desuffotomaculum nigrificans, Desuffotomaculum ruminis, Desuffotomaculum halophilum, Desuffotomaculum acetoxidans, Desuffotomaculum gibsoniae, Desuffotomaculum sapomandens strain, Desuffotomaculum thermosapovorans, Desuffotomaculum, Desuffotomaculum geothermicum, Desuffotomaculum, Desuffosporosinus meridiei, Delta proteobacterium, Thermodesufforhabdus norvegica, Desuffacinum infemum, Desuffacinum hydrothermale, Desufforhabdus amnigena, Desufforhabdus, Desufforhabdus, Desuffomonile tiedjei, Desuffarculus baarsii, Sulfate-reducing bacterium, Sulfate-reducing bacterium, Sulfate-reducing bacterium, Desuffobacterium anilini, Delta proteobacterium, Desuffovibrio profundus strain, Desuffomicrobium baculatum, Desuffocaldus hobo, Desuffovibrio, Desuffovibrio piger, Desuffovibrio ferrophilus, Desuffonatronovibrio hydrogenovorans, Desuffovibrio, Desuffovibrio acrylicus, Desuffovibrio salexigens, Desuffovibrio oxyclinae, Desuffonauticus submarinus, Desuffothermus naphthae, Thermodesuffobacterium, Thermodesuffobacterium hveragerdense, Thermodesuffobacterium thermophilum, Thermodesuffatator indicus, Thermodesuffovibrio yellowstonii, Desuffosporosinus orientis, Desuffotomaculum thermobenzoicum, Desuffotomaculum, Desuffotomaculum, Desuffotomaculum soffataricum, Desuffotomaculum luciae strain, Desuffobacca acetoxidans, Desuffovibrio vulgaris, Desuffovibrio desuffuricans, Desuffovibrio alaskensis, Desuffovibrio magneticus, Desuffosporosinus acidiphilus, Desuffotomaculum kuznetsovii, Desuffotomaculum kuznetsovii, Desuffovibrio suffodismutans, Desuffomicrobium baculatum, Desuffonatronum lacustre, Desuffohalobium retbaense, Desuffonauticus autotrophicus, Thermodesuffobacterium commune, Thermodesuffobacterium hveragerdense, Thermodesuffovibrio islandicus, Thermodesuffovibrio, Thermodesuffobacterium, Desuffotomaculum thermobenzoicum, Desuffotomaculum thermoacetoxidans, Desuffotomaculum thermocistemum, Desuffotomaculum australicum, Desuffotomaculum kuznetsovii, Desuffovibrio desuffuricans, Desuffovibrio alaskensis, Desuffovibrio vulgaris, Desuffovibrio salexigens, Desuffosporosinus acidiphilus, Desuffosporosinus meridiei, Desuffosporosinus orientis, Desuffotomaculum reducens, and combinations thereof.

10. A method of determining an amount of at least one sulphate-reducing bacteria within a sample; wherein the method comprises:
amplifying at least one nucleic acid of at least one sulfur-reducing bacteria in the presence of at least one primer to form an amplification product; wherein the amplifying occurs by a PCR amplification method; wherein the at least one nucleic acid is extracted from the sample prior to amplifying the at least one nucleic acid; wherein the at least one primer comprises an essentially identical sequence selected from the group consisting of SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof;
hybridizing the amplification product with a probe specific for a fragment of an alpha subunit of an APS gene; and
detecting a presence of hybridization and a degree of hybridization; wherein the presence of hybridization indicates the presence of the at least one sulfate-reducing bacteria; and wherein the degree of hybridization enumerates the at least one sulfate-reducing bacteria.

11. The method of claim 10, wherein the probe comprises a nucleotide sequence essentially identical to SEQ ID NO: 16, SEQ ID NO: 17, SEQ ID NO: 18, SEQ ID NO: 19, and mixtures thereof.

12. The method of claim 10, wherein the probe is detectably labeled.

13. The method of claim 10, wherein the at least one primer is specific for amplification of at least a fragment of an alpha subunit of an APS reductase gene.

14. The method of claim 10, wherein the sample is selected from the group consisting of a food product, an animal tissue, a human tissue, a water sample, a lab surface, a metal surface, a paper mill industry sample, a waste water sample from a wastewater treatment facility, a sample from the paint industry, and combinations thereof.

15. The method of claim 10, further comprising extracting the at least one nucleic acid from the sample prior to amplifying at least one nucleic acid.

16. The method of claim 10, wherein the at least one sulfur-species bacteria is selected from the group consisting of Desuffovibrio vulgaris, Desuffovibrio desuffuricans, Desuffovibrio aespoeensis, Thermodesuffobium narugense, Desuffotomaculum carboxydivorans, Desuffotomaculum ruminis, Desuffovibrio africanus, Desuffovibrio hydrothermalis, Desuffovibrio piezophilus, Desuffobacterium corrodens, Sulfate-reducing bacterium QLNR1, Desuffobacterium catecholicum, Desuffobacterium catecholicum, Desuffobulbus marinus, Desuffobulbus, Desuffobulbus propionicus, Desuffocapsa thiozymogenes, Desuffocapsa suffexigens, Desufforhopalus vacuolatus, Desufforhopalus, Desuffofustis glycolicus strain, Desufforhopalus singaporensis, Desuffobacterium, Desuffobacterium zeppelinii strain, Desuffobacterium autotrophicum, Desuffobacula phenolica, Desuffobacula toluolica Tol2, Sulfate-reducing bacterium JHA1, Desuffospira joergensenii, Desuffobacter, Desuffobacter postgatei, Desuffotignum, Desuffotignum balticum, Desufforegula conservatrix, Desuffocella, Desuffobotulus sapovorans, Desuffofrigus, Desuffonema magnum, Desuffonema limicola, Desuffobacterium indolicum, Desuffosarcina variabilis, Desuffatibacillum, Desuffococcus multivorans, Desuffococcus, Desuffonema ishimotonii, Desuffococcus oleovorans Hxd3, Desuffococcus niacini, Desuffotomaculum, Desuffotomaculum nigrificans, Desuffotomaculum ruminis, Desuffotomaculum halophilum, Desuffotomaculum acetoxidans, Desuffotomaculum gibsoniae, Desuffotomaculum sapomandens strain, Desuffotomaculum thermosapovorans, Desuffotomaculum, Desuffotomaculum geothermicum, Desuffotomaculum, Desuffosporosinus meridiei, Delta proteobacterium, Thermodesufforhabdus norvegica, Desuffacinum infemum, Desuffacinum hydrothermale, Desufforhabdus amnigena, Desufforhabdus, Desufforhabdus, Desuffomonile tiedjei, Desuffarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

17. A primer for PCR amplification of at least one nucleic acid of at least one sulfur-reducing bacteria, wherein the primer comprises an essentially identical sequence selected from the group consisting of SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and mixtures thereof.

18. A probe for hybridizing with a PCR amplification product, wherein the probe comprises a nucleotide sequence essentially identical to SEQ ID NO: 16, SEQ ID NO: 17, SEQ ID NO: 18, SEQ ID NO: 19, and mixtures thereof.
